(19) United States
(12) Patent Application Publication
(10) Pub. No.: US 2002/0013357 A1
(43) Pub. Date: Jan. 31, 2002

(54) Valdecoxib compositions

(76) Inventors: Sreekant Nadkarni, Gurnee, IL (US); Mark J. Kontny, Libertyville, IL (US)

Correspondence Address: Pharmacia Corporation, Patent Department Central, 1820, P.O. Box 5110, Chicago, IL, 60680-5110, US

(21) Appl. No.: 09/731,349

(22) Filed: Dec. 6, 2000

Related U.S. Application Data
(60) Provisional application No. 60/169,856, filed on Dec. 9, 1999.
(60) Provisional application No. 60/181,635, filed on Feb. 10, 2000.
(60) Provisional application No. 60/202,269, filed on May 5, 2000.

(51) Int. Cl. A61K 31/415; A61K 9/22

(52) U.S. Cl. 514/406; 424/468

(57) ABSTRACT

Pharmaceutical compositions are provided comprising particulate valdecoxib in an amount of about 1 mg to about 100 mg and one or more pharmaceutically acceptable excipients. The compositions are useful in treatment or prophylaxis of cyclooxygenase-2 mediated conditions and disorders.

This application claims priority of U.S. provisional application Ser. No. 60/169,856 filed on Dec. 9, 1999, U.S. provisional application Ser. No. 60/181,635 filed on Feb. 10, 2000, and U.S. provisional application Ser. No. 60/202,269 filed on May 5, 2000.

### FIELD OF THE INVENTION

The present invention relates to orally deliverable pharmaceutical compositions containing valdecoxib as an active ingredient, to processes for preparing such compositions, to methods of treatment of cyclooxygenase-2 mediated disorders comprising orally administering such compositions to a subject, and to use of such compositions in manufacture of medicaments.

### BACKGROUND OF THE INVENTION

The compound 4-(5-methyl-3-phenyl-4-isoxazolyl)benzenesulfonamide, also referred to herein as valdecoxib, was disclosed in U.S. Pat. No. 5,633,272 to Talley et al. together with processes for preparing this and related compounds. Valdecoxib has the structure:

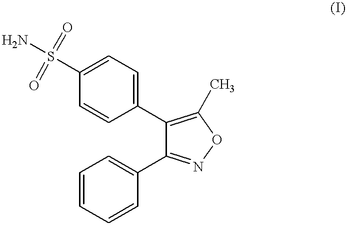

The compounds reported in above-cited U.S. Pat. No. 5,633,272, including valdecoxib, are disclosed therein as useful anti-inflammatory, analgesic and antipyretic drugs having a high degree of selectivity for inhibition of cyclooxygenase-2 (COX-2) over cyclooxygenase-1 (COX-1). Above-cited U.S. Pat. No. 5,633,272 also contains general references to formulations for the administration of such compounds, including orally deliverable dosage forms such as tablets and capsules.

European Patent Application No. 0 863 134 discloses orally deliverable compositions comprising a selective COX-2 inhibitory drug, specifically 2-(3,5-difluorophenyl)-3-(4-methyl-sulfonyl)phenyl)-2-cyclopenten-1-one, in combination with excipient ingredients including microcrystalline cellulose, lactose monohydrate, hydroxypropylcellulose, croscarmellose sodium and magnesium stearate.

International Patent Publication No. WO 00/32189 discloses orally deliverable compositions comprising a selective COX-2 inhibitory drug, specifically celecoxib, in combination with excipient ingredients selected from extensive lists of suitable diluents, disintegrants, binding agents, wetting agents, lubricants, etc.

Valdecoxib has extremely low solubility in water, and for this reason it has been proposed to administer parenterally a much more soluble prodrug, parecoxib, that cleaves to form valdecoxib. See for example Dionne (1999), “COX-2 inhibitors—IBC Conference, 12-13 April 1999, Coronado, Calif., U.S.A.”, IDrugs, 2(7), 664-666.

However, it would be beneficial to have an orally deliverable dosage form of valdecoxib that exhibits good bioavailability and immediate-release properties.

As is indicated hereinbelow, valdecoxib administration is indicated or potentially indicated in a very wide array of COX-2 mediated conditions and disorders. It would therefore be of great benefit to provide orally deliverable formulations having bioavailability characteristics tailored to such indications. It would be of especial benefit to provide immediate-release oral formulations exhibiting pharmacokinetics consistent with a rapid onset effect.

Such formulations would represent a significant advance in the treatment of COX-2 mediated conditions and disorders.

### SUMMARY OF THE INVENTION

There is now provided a pharmaceutical composition comprising particulate valdecoxib in an amount of about 1 mg to about 100 mg per dose and one or more pharmaceutically acceptable excipients.

In one embodiment, a single dose, upon oral administration to a fasting subject, provides a time course of blood serum concentration of valdecoxib having at least one of the following:

(a) a time to reach a threshold concentration for therapeutic effect not greater than about 0.5 h after administration;

(b) a time to reach maximum concentration (Tₘₐₓ) not greater than about 5 h after administration; and

(c) a maximum concentration (Cₘₐₓ) not less than about 100 ng/ml.

By “a threshold concentration for therapeutic effect” is meant a minimum concentration of valdecoxib in blood serum consistent with therapeutic benefit for the particular indication for which the valdecoxib is administered. Typically this threshold concentration is at least about 20 ng/ml, for example about 25 to about 75 ng/ml.

The composition can be in the form of discrete solid articles such as tablets, pills, hard or soft capsules, lozenges, sachets or pastilles, one to a small plurality of which constitute a single dose; alternatively the composition can be in the form of a substantially homogeneous flowable mass, such as a particulate or granular solid or a liquid suspension, from which single doses are measurably removable.

In a presently preferred embodiment, the composition is in the form of tablets wherein the excipients include a water-soluble diluent, a disintegrant, a binding agent and a lubricant. Most preferably the binding agent comprises pregelatinized starch.

Also provided is a method of treating a medical condition or disorder in a subject where treatment with a COX-2 inhibitor is indicated, comprising orally administering a composition of the invention one to about four times a day.

Other features of the invention will be in part apparent and in part pointed out hereinafter.

### BRIEF DESCRIPTION OF THE DRAWINGS

FIG. 1 is a flow diagram illustrating a representative method for preparation of valdecoxib tablets of the invention.

FIG. 2 is a flow diagram illustrating an alternative method for preparation of valdecoxib tablets of the invention.

FIG. 3 is a graph showing plasma concentration of valdecoxib in dogs following oral administration of valdecoxib tablets of the invention.

FIG. 4 is a graph showing plasma concentration of valdecoxib in humans following oral administration of valdecoxib tablets of the invention.

### DETAILED DESCRIPTION OF THE INVENTION

A composition of the invention comprises particulate valdecoxib in a dosage amount of about 1 mg to about 100 mg. Such a composition is a superior immediate-release dosage form capable of providing rapid relief from a COX-2 mediated disorder when orally administered to a subject, more particularly a human subject, suffering from such a disorder.

It is believed, without being bound by theory, that the strong clinical benefits afforded by a composition of the invention result from improved bioavailability of valdecoxib, in particular from surprisingly effective absorption of valdecoxib in the gastrointestinal tract when adminiatered orally in such a composition. Such effective absorption can be verified by one of skill in the art by monitoring blood serum concentration of valdecoxib in a treated subject for a period of time following administration. It is desired to reach, in as short a time as possible, a threshold of valdecoxib concentration in the blood serum consistent with effective COX-2 inhibition.

As indicated above, in one embodiment a single dose, upon oral administration to a fasting subject, provides a time course of blood serum concentration of valdecoxib having at least one of the following:

(a) a time to reach a threshold concentration for therapeutic effect (typically at least about 20 ng/ml) not greater than about 0.5 h after administration;

(b) a time to reach maximum concentration (Tₘₐₓ) not greater than about 5 h after administration; and

(c) a maximum concentration (Cₘₐₓ) not less than about 100 ng/ml.

It will be understood that the amount of valdecoxib in a dose unit effective to provide blood serum concentrations meeting any of criteria (a) to (c) immediately above is dependent on the body weight of the treated subject. Where the subject is a child or a small animal (e.g., a dog), for example, an amount of valdecoxib relatively low in the indicated range of about 1 mg to about 100 mg is likely to provide blood serum concentrations consistent with at least one of criteria (a) to (c). Where the subject is an adult human or a large animal (e.g., a horse), the indicated blood serum concentrations of valdecoxib are likely to require a relatively greater dosage amount of valdecoxib. For an adult human, a suitable amount of valdecoxib per dose in a composition of the present invention to provide the indicated blood serum concentrations is typically about 5 mg to about 40 mg.

In a preferred embodiment, the bioavailability of the composition is such that, when a 20 mg dose is administered orally to a fasting adult human subject:

(a) a valdecoxib blood serum concentration of 20 ng/ml, more preferably of 50 ng/ml, is reached not more than about 0.5 h after administration;

(b) Tₘₐₓ is not greater than about 3 h after administration; and

(c) Cₘₐₓ is not less than about 100 ng/ml.

Compositions of the invention contain valdecoxib in particulate form. Primary valdecoxib particles, generated for example by milling or grinding, or by precipitation from solution, can agglomerate to form secondary aggregate particles. The term “particle size” as used herein refers to size, in the longest dimension, of primary particles, unless the context demands otherwise. Particle size is believed to be an important parameter affecting clinical effectiveness of valdecoxib. Thus, in one embodiment, a composition has a distribution of valdecoxib particle sizes such that the D₉₀ particle size is less than about 75 μm. The “D₉₀ particle size” is defined herein as a particle size such that 90% by weight of the particles are smaller, in their longest dimension, than that particle size.

In addition or alternatively, valdecoxib particles in a composition of the invention preferably have a weight average particle size of about 1 μm to about 10 μm, most preferably about 5 μm to about 7 μm.

Compositions of the invention comprise valdecoxib together with one or more excipients selected from diluents, disintegrants, binding agents, wetting agents and lubricants. In one preferred embodiment at least one of the excipients is a water-soluble diluent or wetting agent. Such a water-soluble diluent or wetting agent is believed to assist in dispersion and dissolution of the valdecoxib in the gastrointestinal tract. Preferably at least a water-soluble diluent is present. In another preferred embodiment at least one of the excipients is a disintegrant. In another preferred embodiment at least one of the excipients is a binding agent; as indicated above, it is particularly preferred that pregelatinized starch be present as a binding agent. In another preferred embodiment at least one of the excipients is a lubricant. It is esepcially preferred that the composition comprise, in addition to valdecoxib, each of a water-soluble diluent, a disintegrant, a binding agent and a lubricant.

A composition of the invention can be a substantially homogeneous flowable mass such as a particulate or granular solid or a liquid, or it can be in the form of discrete articles such as capsules or tablets. in a composition that is a substantially homogeneous flowable mass, single doses are measurably removable using a suitable volumetric measuring device such as a spoon or cup. Suitable flowable masses include, but are not limited to, powders and granules. Alternatively, the flowable mass can be a suspension having the valdecoxib in a solid particulate phase dispersed in a liquid phase, preferably an aqueous phase. In preparing such a suspension, use of a wetting agent such as polysorbate 80 or the like is likely to be beneficial. A suspension can be prepared by dispersing milled valdecoxib in the liquid phase; alternatively the valdecoxib can be precipitated from solution in a solvent such as an alcohol, preferably ethanol. The aqueous phase preferably comprises a palatable vehicle such as water, syrup or fruit juice, for example apple juice.

Compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects than compositions of conventional nonsteroidal anti-inflammatory drugs (NSAIDs) that lack selectivity for COX-2 over COX-1. In particular, compositions of the invention have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendinitis, bursitis, allergic neuritis, cytomegalovirus infectivity, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, conjunctivitis, retinopathies, uveitis, ocular photophobia, and of acute injury to the eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

Such compositions are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophogeal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

Such compositions inhibit prostanoid-induced smooth muscle contraction by inhibiting synthesis of contractile prostanoids and hence can be of use in treatment of dysmenorrhea, premature labour, asthma and eosinophil-related disorders. They also can be of use for decreasing bone loss particularly in postmenopausal women (i.e., treatment of osteoporosis), and for treatment of glaucoma.

Preferred uses for compositions of the invention are for treatment of rheumatoid arthritis and osteoarthritis, for pain management generally (particularly post-oral surgery pain, post-general surgery pain, post-orthopedic surgery pain, and acute flares of osteoarthritis), for treatment of Alzheimer's disease, and for colon cancer chemoprevention.

Besides being useful for human treatment, compositions of the invention are useful for veterinary treatment of companion animals, exotic animals, farm animals, and the like, particularly mammals. More particularly, compositions of the invention are useful for treatment of COX-2 mediated disorders in horses, dogs and cats.

The present invention is further directed to a therapeutic method of treating a condition or disorder where treatment with a COX-2 inhibitory drug is indicated, the method comprising oral administration of a composition of the invention to a subject in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely and can therefore deviate from the preferred dosage regimens set forth above.

Initial treatment can begin with a dose regimen as indicated above. Treatment is generally continued as necessary over a period of several weeks to several months or years until the condition or disorder has been controlled or eliminated. Subjects undergoing treatment with a composition of the invention can be routinely monitored by any of the methods well known in the art to determine effectiveness of therapy. Continuous analysis of data from such monitoring permits modification of the treatment regimen during therapy so that optimally effective doses are administered at any point in time, and so that the duration of treatment can be determined. In this way, the treatment regimen and dosing schedule can be rationally modified over the course of therapy so that the lowest amount of the composition exhibiting satisfactory effectiveness is administered, and so that administration is continued only for so long as is necessary to successfully treat the condition or disorder.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lornoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac (see The Merck Index, 12th Edition (1996), Therapeutic Category and Biological Activity Index, lists therein headed “Analgesic”, “Anti-inflammatory” and “Antipyretic”).

Particularly preferred combination therapies comprise use of a composition of the invention with an opioid compound, more particularly where the opioid compound is codeine, meperidine, morphine or a derivative thereof.

A valdecoxib composition of the invention can also be administered in combination with a second selective COX-2 inhibitory drug, for example celecoxib, rofecoxib, etc.

The compound to be administered in combination with valdecoxib can be formulated separately from the valdecoxib or co-formulated with the valdecoxib in a composition of the invention. Where valdecoxib is co-formulated with a second drug, for example an opioid drug, the second drug can be formulated in immediate-release, rapid-onset, sustained-release or dual-release form.

Compositions of the invention are generally suitable for administration of valdecoxib in a daily dosage amount from about 1 mg to about 100 mg. Each dose unit of a composition of the invention typically comprises an amount of valdecoxib from about one-tenth of the daily dosage amount to the whole of a daily dosage amount. Preferred daily dosage amounts are about 2 mg to about 60 mg, more preferably about 5 mg to about 40 mg, for example about 5 mg, about 10 mg, about 20 mg or about 40 mg. Where the dose units are in the form of discrete articles suitable for oral administration, such as capsules or tablets, each such article comprises about 1 mg to about 100 mg, preferably about 5 mg to about 60 mg, more preferably about 10 mg to about 50 mg, for example about 10 mg, about 20 mg or about 40 mg, of valdecoxib.

The valdecoxib used in compositions of the invention can be prepared by any process known per se, including in the manner set forth in above-cited U.S. Pat. No. 5,633,272.

In addition to valdecoxib, compositions of the invention comprise one or more excipients suitable for oral administration. The excipients must be pharmaceutically acceptable in the sense of being compatible with the other ingredients of the composition and must not be deleterious to the recipient. Excipients employed can be solids or liquids, or both.

A composition of the invention contains a desired amount of valdecoxib per dose and can be in the form of, for example, a tablet, a pill, a hard or soft capsule, a lozenge, a cachet, a dispensable powder, granules, a suspension, or any other form reasonably adapted for oral administration. Tablets, pills and the like can be prepared with or without coatings.

Compositions of the invention suitable for buccal or sublingual administration include, for example, lozenges comprising valdecoxib in a flavored base, such as sucrose, and acacia or tragacanth, and pastilles comprising valdecoxib in an inert base such as gelatin and glycerin or sucrose and acacia.

Liquid dosage forms include suspensions of valdecoxib in a liquid diluent, which is typically aqueous. Such suspensions can contain additional excipients, for example wetting agents, emulsifying and suspending agents, stabilizing agents, thickening agents, and sweetening, flavoring, and perfuming agents.

Compositions of the invention can be prepared by any suitable method of pharmacy which includes a step of bringing into association the valdecoxib and the excipient(s). In general, the compositions are prepared by uniformly and intimately admixing valdecoxib with a liquid or finely divided solid diluent, and then, if necessary, encapsulating or shaping the resulting blend. For example, a tablet can be prepared by compressing or molding a powder or granules of such a blend, optionally together with one or more additional excipients. Compressed tablets can be prepared by compressing, in a suitable machine, a free-flowing composition, such as a powder or granules, comprising valdecoxib optionally mixed with one or more diluents, disintegrants, binding agents and lubricants. Molded tablets can be prepared by molding, in a suitable machine, powdered valdecoxib, optionally with one or more excipients, moistened with a liquid diluent.

Through selection and combination of excipients, compositions can be provided exhibiting improved performance with respect to efficacy, bioavailability, clearance time, stability, compatibility of valdecoxib and exceipients, safety, dissolution profile, disintegration profile and/or other pharmacokinetic, chemical and/or physical properties. The excipients preferably include one or more materials that are water-soluble or water-dispersible and have wetting properties to offset the low aqueous solubility and hydrophobicity of valdecoxib. Where the composition is formulated as a tablet, the combination of excipients selected provides tablets that can exhibit improvement, among other properties, in dissolution and disintegration profiles, hardness, crushing strength and/or friability.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility.

Lactose and microcrystalline cellulose, either individually or in combination, are preferred diluents. Both diluents are chemically compatible with valdecoxib. The use of extragranular microcrystalline cellulose (that is, microcrystalline cellulose added to a wet granulated composition after a drying step) can be used to improve hardness (for tablets) and/or disintegration time. Lactose, especially lactose monohydrate, is particularly preferred. Lactose typically provides compositions having suitable release rates of valdecoxib, stability, pre-compression flowability, and/or drying properties at a relatively low diluent cost. It provides a high density substrate that aids densification during granulation (where wet granulation is employed) and therefore improves blend flow properties.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscannellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated compositions of the present invention.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and sodium carboxymethylcellulose (e.g., Tylose™); alginic acid and salts of alginic acid; magnesium aluminum silicate; polyethylene glycol (PEG); guar gum; polysaccharide acids; bentonites; polyvinylpyrrolidone (povidone or PVP), for example povidone K-15, K-30 and K-29/32; polymethacrylates; hydroxypropylmethylcellulose (HPMC); hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

Pregelatinized starch is a preferred binding agent used to impart cohesive properties to a powder blend of valdecoxib and other excipients for granulation of a valdecoxib formulation. Pregelatinized starch, if present, preferably constitutes about 0.5% to about 20%, more preferably about 5% to about 15%, of the total weight of the composition, and facilitates binding of particles in the blend to form granules during wet granulation.

Compositions of the invention optionally comprise one or more pharmaceutically acceptable wetting agents as excipients. Such wetting agents are preferably selected to maintain the valdecoxib in close association with water, a condition that is believed to improve bioavailability of the composition.

Non-limiting examples of surfactants that can be used as wetting agents in compositions of the present invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; polyethylene glycols (e.g., Carbowax™ 4000 and Carbowax™ 6000); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the composition.

Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the composition.

Other excipients such as colorants, flavors and sweeteners are known in the pharmaceutical art and can be used in compositions of the present invention. Tablets can be coated, for example with an enteric coating, or uncoated. Compositions of the invention can further comprise, for example, buffering agents.

Particle size reduction of the valdecoxib can lead to improved bioavailability when the drug is formulated as an orally deliverable composition in accordance with the invention. Accordingly, the D₉₀ particle size of the valdecoxib is preferably less than about 75 μm, even more preferably less than about 40 μm, and most preferably less than about 25 μm. In addition or alternatively, the valdecoxib preferably has a weight average particle size in the range of about 1 μm to about 10 μm, more preferably about 5 μm to about 7 μm. Any suitable milling, grinding or micronizing method can be used for particle size reduction.

Capsule and tablet compositions of the invention are immediate release compositions that release at least about 50%, more preferably at least about 60% and most preferably at least about 75% of the valdecoxib, as measured in vitro in a standard dissolution assay, within about 45 minutes.

Especially preferred capsule and tablet compositions of the invention release in vitro at least about 50% of the valdecoxib within about 15 minutes, and/or at least about 60% of the valdecoxib within about 30 minutes.

Although compositions of the invention can be prepared, for example, by direct encapsulation or direct compression, they are preferably wet granulated prior to encapsulation or compression. Wet granulation, among other effects, densifies milled compositions resulting in improved flow properties, improved compression characteristics and easier metering or weight dispensing of the compositions for encapsulation or tableting. The secondary particle size resulting from granulation (i.e., granule size) is not narrowly critical, it being important only that the average granule size preferably is such as to allow for convenient handling and processing and, in the case of tablets, to permit formation of a readily compressible mixture that forms pharmaceutically acceptable tablets.

Desired bulk density of the granules when poured or tapped is normally about 0.3 to about 1.0 g/ml, for example about 0.6 to about 0.9 g/ml.

To prepare tablets by compression, the granulated blend in an amount sufficient to make a uniform batch of tablets can be processed in a conventional production scale tableting machine at normal compression pressure (for example, applying a force of about 1 to about 50 kN in a typical tableting die). The resulting tablet hardness should be convenient with respect to handling, manufacture, storage and ingestion may be employed; however a minimum hardness of about 4 kP, preferably about 5 kP and more preferably about 6 kP, is desirable to avoid excessive friability, and a maximum hardness of about 18 kP, preferably about 15 kP and more preferably about 12 kP, is desirable to avoid subsequent difficulty in hydrating the tablet when exposed to gastric fluid. When hardness is in an acceptable range, tablet friability is typically less than about 1.0%, preferably less than about 0.8% and more preferably less than about 0.5%, in a standard test.

Excipients, in particular a disintegrant, for immediate release capsule and tablet compositions of the invention are preferably selected to provide a disintegration time in a standard in vitro assay of less than about 30 minutes, preferably less than about 25 minutes, more preferably less than about 20 minutes, and still more preferably less than about 15 minutes.

The invention is further directed to methods for preparation of compositions comprising particulate valdecoxib. In a particular embodiment, the invention is directed to methods for preparation of such compositions in the form of tablets. Although dry granulation or direct compression methods can be used, methods comprising a wet granulation step are presently preferred. In two illustrative embodiments, wet granulation is performed under low and high shear respectively.

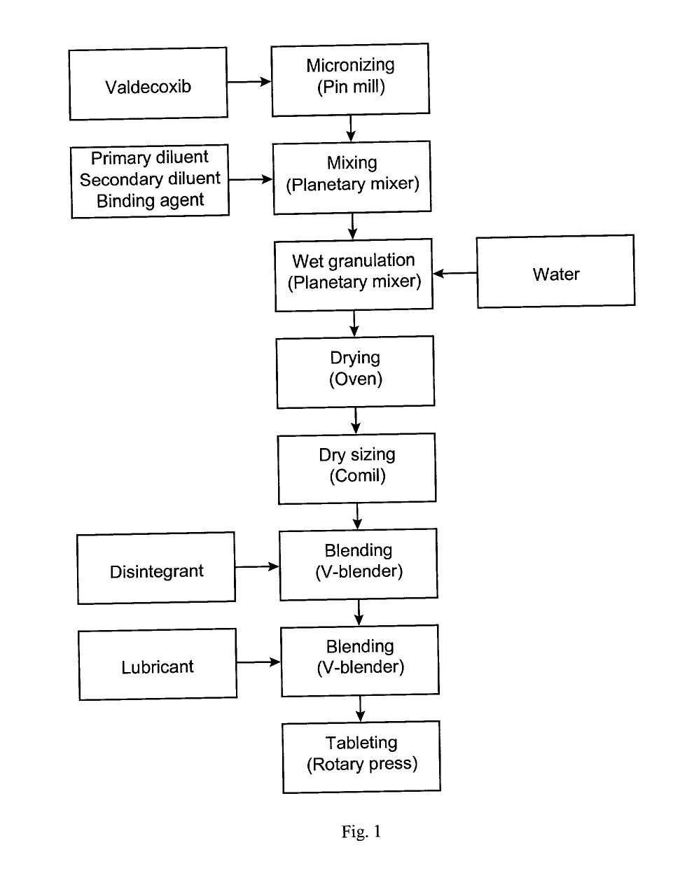

A low shear process is outlined diagrammatically in FIG. 1. In this illustrative process, micronized valdecoxib is mixed, for example in a planetary mixer, with one or more solid particulate diluents, e.g., lactose monohydrate (primary diluent) and microcrystalline cellulose (secondary diluent), and a binding agent, preferably pregelatinized starch, to form a premix. Water is then added, with continued mixing, in an amount to promote formation of granules. The granules are dried, for example in an oven, and then sized in a comil with appropriate screen size to provide fairly uniform granules. These are then blended with a disintegrant, e.g., croscarmellose sodium, and finally with a lubricant, e.g., magnesium stearate, to produce a tableting blend. It will be noted that in this illustrative process, microcrystalline cellulose is added intragranularly and croscarmellose sodium extragranularly. Finally, the tableting blend is compressed, for example in a rotary press, to form tablets. The tablets can optionally be coated using any suitable coating process known in the art.

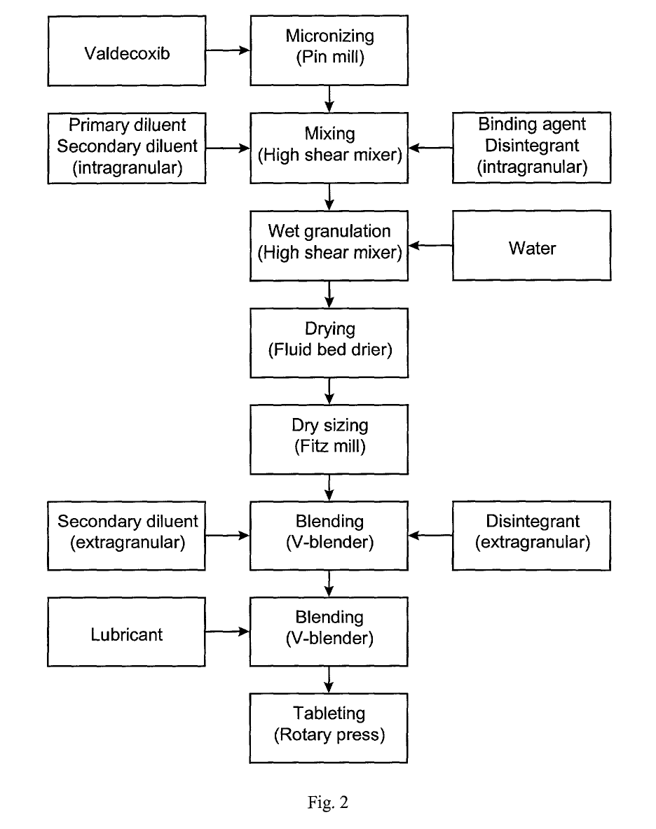

A high shear process is outlined diagrammatically in FIG. 2. In this illustrative process, micronized valdecoxib is mixed in a high shear mixer with a primary diluent, e.g., lactose monohydrate, a first portion of a secondary diluent, e.g., microcrystalline cellulose, a binding agent, preferably pregelatinized starch, and a first portion of a disintegrant, e.g., croscarmellose sodium, to form a premix. Water is then added, with continued high shear mixing, in an amount to promote formation of granules. The granules are optionally wet sized and then dried, preferably in a fluid bed drier. A dry sizing step, for example in a Fitz mill, can then be conducted. The resulting granules are then blended with a second portion of the secondary diluent and a second portion of the disintegrant, and finally with a lubricant, e.g., magnesium stearate, to produce a tableting blend. It will be noted that in this illustrative process, microcrystalline cellulose and croscarmellose sodium are each added both intragranularly and extragranularly. Finally, the tableting blend is compressed and optionally coated, as in the low shear process.

The present invention also is directed to use of compositions of the present invention in preparation of medicaments useful in treatment and/or prophylaxis of COX-2 mediated conditions and disorders.

### EXAMPLES

The following examples illustrate aspects of the present invention but should not be construed as limitations. Unless otherwise stated, all percentages recited in these examples are by weight based on total composition weight.

### Example 1

### Valdecoxib 10 mg Tablets Prepared by Low Shear Wet Granulation

Tablets were prepared having the composition shown in Table 1.

TABLE 1

| Component | Function | Amount (mg) |
|---|---|---|
| valdecoxib, micronized | active ingredient | 10 |
| lactose monohydrate NF, #310 | primary diluent | 105 |
| microcrystalline cellulose NF | secondary diluent | 60 |
| (Avicel ™ PH-101) | | |
| pregelatinized starch NF | binding agent | 20 |
| (National Starch 1500) | | |
| croscarmellose sodium NF | disintegrant | 4 |
| (Ac-Di-Sol ™) | | |
| magnesium stearate | lubricant | 1 |
| Total tablet weight | | 200 |

The appropriate amount of micronized valdecoxib for the batch size was first mixed with an equal amount of lactose monohydrate, screened by passing through a 20 mesh screen, and added to a Hobart planetary mixer. The balance of the lactose monohydrate and the microcrystallized cellulose were then added to the mixer, which was then operated at a slow impeller speed for about 10 minutes. The resulting premix was then granulated in the planetary mixer by adding purified water manually over 12-15 minutes while continuing to mix at a slow to medium impeller speed. The resulting wet granules were dried on trays in a Gruenberg oven with an inlet air temperature of 60±5° C. to a moisture content of 2.0±1.0%, measured by loss on drying. The resulting dry granules were sized through a size 14 screen using a Quadro comil at medium speed, and then placed in a Patterson Kelley V-blender together with the croscarmellose sodium. The V-blender was operated for about 5 minutes to thoroughly mix the croscarmellose sodium with the granules; then magnesium stearate was added with further mixing for about 3 minutes to prepare a lubricated blend. This was compressed on a Manesty DB16 rotary press using 7.5 mm standard concave tooling to provide a tablet weight of 200±10 mg having a hardness of 10±4 kP.

### Example 2

### Valdecoxib 10 mg Tablets Prepared by High Shear Wet Granulation

Tablets were prepared having the composition shown in Table 2.

TABLE 2

| Component | Function | Amount (mg) |
|---|---|---|
| valdecoxib, micronized | active ingredient | 10 |
| lactose monohydrate NF, #310 | primary diluent | 103 |
| microcrystalline cellulose NF | secondary diluent | 60 |
| (Avicel ® PH-101) | | |
| intragranular | | 30 |
| extragranular | | 30 |
| pregelatinized starch NF | binding agent | 20 |
| (National Starch 1500) | | |
| croscarmellose sodium NF | disintegrant | 6 |
| (Ac-Di-Sol ®) | | |
| intragranular | | 3 |
| extragranular | | 3 |
| magnesium stearate | lubricant | 1 |
| Total tablet weight | | 200 |

The micronized valdecoxib, lactose monohydrate, intragranular microcrystalline cellulose, pregelatinized starch and intragranular croscarmellose sodium were mixed in a Baker Perkins high shear mixer at high impeller/chopper speed for about 3 minutes to form a premix. Purified water was added to the premix via a Watson Marlow peristaltic pump over a period of about 3 minutes and mixing continued for a further 45 seconds. The resulting wet granules were dried in an Aeromatic fluid bed drier with an inlet air temperature of 60±5° C. to a moisture content of 2.0±1.0% as measured by loss on drying, to form a dry granulate. The dry granulate was sized through a 20 mesh screen using a Fitz mill with knives forward, at 1800 rpm, and was then placed in a Patterson Kelley V-blender. Here, the granulate was mixed with the extragranular microcrystalline cellulose and extragranular croscarmellose sodium for about 5 minutes, and then with the magnesium stearate for a further 3 minutes, to form a lubricated blend. This was compressed on a Korsch PH-230 rotary press using 7.5 mm standard concave tooling to provide a tablet weight of 200±10 mg. Tablets were prepared having hardnesses of 6, 8, 10 and 12 kP.

### Example 3

### Coated Valdecoxib 5, 10, 20 and 40 mg Tablets

Using the process of Example 2, tablets were prepared having the composition shown in Table 3. Tablets were film coated with Opadry Yellow YS-1-12525A or Opadry White YS-1-18027A at 3% of uncoated tablet weight, using a 15% suspension of the coating material in water.

TABLE 3

| Ingredient | Amount/tablet (mg) | | | |
|---|---|---|---|---|
| valdecoxib, micronized | 5 | 10 | 20 | 40 |
| lactose monohydrate NF | 108 | 103 | 206 | 186 |
| microcrystalline cellulose NF | 60 | 60 | 120 | 120 |
| pregelatinized starch NF | 20 | 20 | 40 | 40 |
| croscarmellose sodium NF | 6 | 6 | 12 | 12 |
| magnesium stearate NF | 1 | 1 | 2 | 2 |
| Total weight (excluding coating) | 200 | 200 | 400 | 400 |
| Opadry Yellow YS-1-12525A | 6 | | | 12 |
| Opadry White YS-1-18027A | | 6 | 12 | |

Properties of the tablets of Example 3 are presented in Table 4.

Disintegration was evaluated by the following procedure. Six identical tablets were separately placed into one of six tubes having a wire mesh screen bottom in a disintegration basket. A water bath was preheated to 37° C.±2° C. and maintained at that temperature for the duration of the disintegration test. A 1000 ml beaker was placed in the water bath. The beaker was filled with a sufficient amount of water to ensure that the wire mesh screen of the tubes would remain at least 2.5 cm below the water surface during the test. The disintegration basket was inserted in the water and repeatedly raised and lowered until the test was complete while maintaining the wire mesh screen of the tubes at least 2.5 cm below the water surface. Disintegration time for each tablet was the time, measured from time of insertion of the basket, at which the very last portion of the tablet passed through the screen at the bottom of the tube.

TABLE 4

| | 5 mg | 10 mg | 20 mg | 40 mg |
|---|---|---|---|---|
| Shape | oval | caplet | caplet | heptagon |
| Thickness | 3.6 ± 0.2 | 3.6 ± 0.2 | 4.8 ± 0.4 | 4.2 ± 0.3 |
| (mm) | | | | |
| Hardness (kP) | 9 ± 3 | 9 ± 3 | 13 ± 5 | 13 ± 5 |
| Friability (%) | <0.8 | <0.8 | <0.8 | <0.8 |
| Disintegration | 12 minutes | 12 minutes | 12 minutes | 12 minutes |
| in vitro | | | | |

### Example 4

### Pharmacokinetic Properties of Valdecoxib Tablets in Dogs

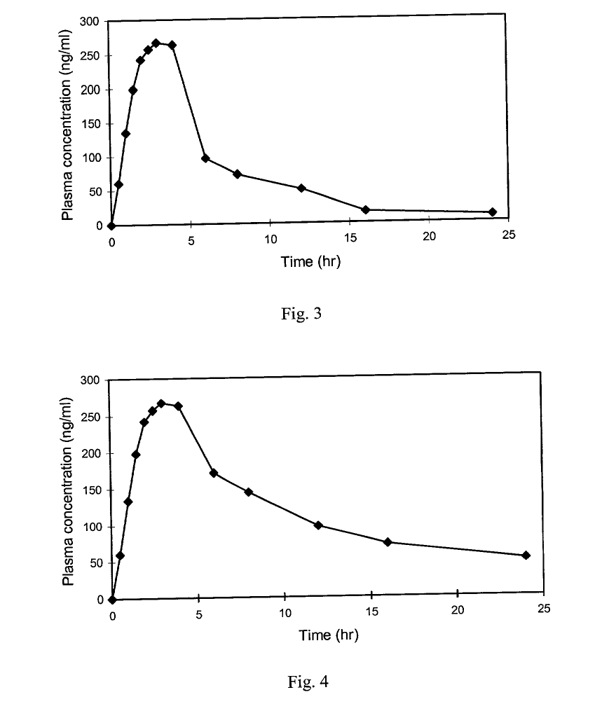

A study was performed in order to determine pharmacokinetic properties of the valdecoxib composition of Example 2, in 23 beagle dogs. Valdecoxib was administered at a dose of 20 mg (2 tablets). Venous blood was collected pre-dose, and at 0.5, 1, 1.5, 2, 2.5, 3, 4, 6, 8, 12 and 24 hours after oral dose administration. Plasma was separated from blood by centrifugation at 3000 G and samples were stored at −20° C. until analysis. Concentrations of valdecoxib in plasma were determined using an HPLC assay. Results are shown in FIG. 3.

### Example 5

### Pharmacokinetic Properties of Valdecoxib Tablets in Humans

A study was performed in order to determine pharmacokinetic properties of the valdecoxib composition of Example 2, in 24 healthy adult humans. Valdecoxib was administered at a dose of 20 mg (2 tablets). Venous blood was collected pre-dose, and at 0.5, 1, 1.5, 2, 2.5, 3, 4, 6, 8, 12, 16 and 24 hours after oral dose administration. Plasma was separated from blood by centrifugation at 3000 G and samples were stored at −20° C. until analysis. Concentrations of valdecoxib in plasma were determined using an HPLC assay. Results are shown in FIG. 4.

Calculated Cₘₐₓ was 303±93 ng/ml. Calculated Tₘₐₓ was 2.97±0.73 h.

What is claimed is:

1. A pharmaceutical composition comprising particulate valdecoxib in an amount of about 1 mg to about 100 mg per dose and one or more pharmaceutically acceptable excipients, wherein a single dose, upon oral administration to a fasting subject, provides a time course of blood serum concentration of valdecoxib having at least one of
(a) a time to reach a threshold concentration for therapeutic effect not greater than about 0.5 h after administration;
(b) a time to reach maximum concentration (Tₘₐₓ) not greater than about 5 h after administration; and
(c) a maximum concentration (Cₘₐₓ) not less than about 100 ng/ml.

2. The composition of claim 1 wherein the threshold concentration for therapeutic effect is about 20 ng/ml.

3. The composition of claim 2 wherein a single dose, upon oral administration to a fasting subject, provides a time course of blood serum concentration of valdecoxib having each of
(a) a time to reach a concentration of 20 ng/ml not greater than about 0.5 h after administration;
(b) a time to reach maximum concentration (Tₘₐₓ) not greater than about 3 h after administration; and
(c) a maximum concentration (Cₘₐₓ) not less than about 100 ng/ml.

4. The composition of claim 1 wherein the valdecoxib is in an amount of about 5 mg to about 40 mg per dose.

5. The composition of claim 1 that is a tablet wherein the excipients comprise one or more diluents in an amount of about 5% to about 99%, one or more disintegrants in an amount of about 0.2% to about 30%, one or more binding agents in an amount of about 0.5% to about 25%, and one or more lubricants in an amount of about 0.1% to about 10%, by weight of the composition.

6. The composition of claim 5 wherein the binding agent is pregelatinized starch.

7. The composition of claim 1 that is a tablet wherein the excipients comprise lactose monohydrate, microcrystalline cellulose, croscarmellose sodium, pregelatinized starch and magnesium stearate.

8. The composition of claim 1 further comprising one or more opioid or analgesic drugs.

9. The composition of claim 1 wherein D₉₀ of the valdecoxib particles is less than about 75 μm.

10. The composition of claim 1 wherein the valdecoxib particles have a weight average particle size of about 1 to about 10 μm.

11. A process for preparing a composition of claim 5 comprising a step of wet granulating valdecoxib together with one or more diluents and a binding agent, a step of drying the resulting granules and a step of compressing the resulting dry granulate to form a tablet.

12. The process of claim 11 wherein, prior to the wet granulating step, valdecoxib is mixed under low shear with one or more diluents and a binding agent to form a premix for wet granulation; and wherein, between the drying step and the compressing step, the granules are blended with a disintegrant and a lubricant to form a blend for tableting.

13. The process of claim 12 wherein the binding agent is pregelatinized starch.

14. The process of claim 13 wherein the diluents comprise lactose monohydrate and microcrystalline cellulose, the disintegrant is croscarmellose sodium and the lubricant is magnesium stearate.

15. The process of claim 11 wherein, prior to the wet granulating step, valdecoxib is mixed under high shear with a primary diluent, a first portion of a secondary diluent, a binding agent and a first portion of a disintegrant, to form a premix for wet granulation; and wherein, between the drying step and the compressing step, the granules are blended with a second portion of the secondary diluent, a second portion of the disintegrant, and a lubricant, to form a blend for tableting.

16. The process of claim 15 wherein the binding agent is pregelatinized starch.

17. The process of claim 16 wherein the diluents comprise lactose monohydrate and microcrystalline cellulose, the disintegrant is croscarmellose sodium and the lubricant is magnesium stearate.

18. A method of treating a medical condition or disorder in a subject where treatment with a cyclooxygenase-2 inhibitor is indicated, comprising orally administering to the subject a composition of claim 1 once or twice a day.
